(19) United States
(12) Patent Application Publication
(10) Pub. No.: US 2025/0366804 A1
(43) Pub. Date: Dec. 4, 2025

(54) DATA PROCESSING SYSTEM, DATA PROCESSING METHOD, AND DATA PROCESSING PROGRAM

(71) Applicant: FUJIFILM Corporation, Tokyo (JP)

(72) Inventors: Isao TAKAHASHI, Tokyo (JP); Shinichi KOJIMA, Tokyo (JP); Kazuma YOKOI, Tokyo (JP)

(73) Assignee: FUJIFILM Corporation, Tokyo (JP)

(21) Appl. No.: 19/225,097

(22) Filed: Jun. 2, 2025

(30) Foreign Application Priority Data
Jun. 3, 2024 (JP) 2024-090067

(51) Int. Cl. A61B 6/42; A61B 6/03; A61B 6/40

(52) U.S. Cl. CPC A61B 6/4241; A61B 6/032; A61B 6/4035

(57) ABSTRACT

A data processing system includes a first data processing apparatus that processes projection data from a photon counting detector, and a second data processing apparatus. The first data processing apparatus derives at least one piece of first compressed projection data by making resolution of first projection data of at least one energy band among a plurality of pieces of projection data lower than resolution of projection data of a plurality of other energy bands, and transmits the first compressed projection data and the projection data of other energy bands to the second data processing apparatus. The second data processing apparatus acquires the first compressed projection data and the projection data of the plurality of other energy bands, and derives first restored projection data by restoring the first compressed projection data based on the projection data of the plurality of other energy bands and the first compressed projection data.

### CROSS REFERENCE TO RELATED APPLICATIONS

The present application claims priority from Japanese Patent Application No. 2024-090067, filed on Jun. 3, 2024, the entire disclosure of which is incorporated herein by reference.

### BACKGROUND

### Technical Field

The present disclosure relates to a data processing system, a data processing method, and a data processing program.

### 2. Description of the Related Art

In recent years, a photon counting computed tomography (PCCT) apparatus that is a radiography apparatus equipped with a photon counting detector has been known. The PCCT apparatus is capable of obtaining a high-resolution image which is a tomographic image with a higher resolution compared to a tomographic image of a computed tomography (CT) apparatus in the related art, and is also capable of measuring the energy of each photon to acquire energy information of each of a plurality of energy bands. Therefore, the PCCT apparatus is capable of obtaining more information than the CT apparatus in the related art.

On the other hand, the detector is disposed on a rotating plate together with the X-ray source, and it is necessary to transmit a detection signal detected by the detector as projection data to a console while rotating the rotating plate during imaging due to the structure of the PCCT apparatus. Therefore, the projection data is transmitted from the detector to the console through a slip ring instead of a conductive wire.

However, the slip ring has a high level of difficulty in data transmission compared to the conductive wire. For this reason, in order to improve the transmission efficiency by reducing the amount of data, various methods for compressing the projection data output from the detector have been proposed. For example, US2018/0146938A proposes a method of compressing second projection data by reducing a resolution of the second projection data among first projection data and second projection data of two energy bands, a first energy band and a second energy band, transmitting the compressed second projection data and the first projection data, and restoring the second projection data using the first projection data at a transmission destination.

On the other hand, the energy bands that the photon counting detector can measure may be three or more, not only two.

### SUMMARY OF THE INVENTION

The present disclosure has been made in view of the above circumstances, and an object of the present disclosure is to efficiently transmit projection data of three or more energy bands while ensuring reproducibility.

A data processing system according to an aspect of the present disclosure comprises: a first data processing apparatus that processes projection data from a photon counting detector which detects radiation emitted from a radiation source and transmitted through a subject and which outputs a plurality of pieces of projection data assigned to each of three or more energy bands corresponding to the number of photons of the radiation; and a second data processing apparatus, in which the first data processing apparatus includes at least one first processor, the first processor derives at least one piece of first compressed projection data by making resolution of at least one piece of first projection data of at least one energy band among the plurality of pieces of projection data relatively lower than resolution of projection data of a plurality of other energy bands other than the at least one energy band, and transmits the at least one piece of first compressed projection data and the projection data of the plurality of other energy bands to the second data processing apparatus, the second data processing apparatus includes at least one second processor, and the second processor acquires the at least one piece of first compressed projection data and the projection data of the plurality of other energy bands, and derives first restored projection data by restoring the first compressed projection data based on the projection data of the plurality of other energy bands and the first compressed projection data.

In the data processing system according to the present disclosure, an energy band of the first projection data may be relatively higher in energy than an energy band of at least one piece of projection data among the projection data of the plurality of other energy bands.

In addition, the data processing system according to the present disclosure, the second processor may derive first restored compressed projection data by restoring the first compressed projection data based on a relationship between the first projection data and the projection data of the plurality of other energy bands, which is derived in advance, and derive the first restored projection data by correcting the first restored compressed projection data to match the first compressed projection data.

In addition, the data processing system according to the present disclosure, the photon counting detector may output three pieces of projection data assigned to each of three energy bands, the first processor may derive the first compressed projection data from the first projection data among the three pieces of projection data, and transmits second projection data and third projection data as the projection data of the plurality of other energy bands other than the first projection data among the three pieces of projection data together with the first compressed projection data to the second data processing apparatus, and the second processor may derive the first restored compressed projection data by restoring the first compressed projection data based on a relationship between the first projection data, the second projection data, and the third projection data.

In addition, the data processing system according to the present disclosure, the second processor may derive the first restored compressed projection data by restoring the first compressed projection data based on a relationship between a first ratio of the second projection data to the third projection data and a second ratio of the first projection data to the third projection data.

A data processing method according to another aspect of the present disclosure in a data processing system including a first data processing apparatus that processes projection data from a photon counting detector which detects radiation emitted from a radiation source and transmitted through a subject and which outputs a plurality of pieces of projection data assigned to each of three or more energy bands corresponding to the number of photons of the radiation and a second data processing apparatus, the method comprises: causing a computer to, in the first data processing apparatus, derive at least one piece of first compressed projection data by making resolution of at least one piece of first projection data of at least one energy band among the plurality of pieces of projection data relatively lower than resolution of projection data of a plurality of other energy bands other than the at least one energy band, and transmit the at least one piece of first compressed projection data and the projection data of the plurality of other energy bands to the second data processing apparatus.

In the data processing method according to the present disclosure, the method may further comprise causing the computer to, in the second data processing apparatus, acquire the at least one piece of first compressed projection data and the projection data of the plurality of other energy bands transmitted by the data processing method according to the present disclosure, and derive first restored projection data by restoring the first compressed projection data based on the projection data of the plurality of other energy bands and the first compressed projection data.

A data processing program according to still another aspect of the present disclosure that causes a computer to execute data processing in a data processing system including a first data processing apparatus that processes projection data from a photon counting detector which detects radiation emitted from a radiation source and transmitted through a subject and which outputs a plurality of pieces of projection data assigned to each of three or more energy bands corresponding to the number of photons of the radiation and a second data processing apparatus, the data processing program causing the computer to execute: in the first data processing apparatus, a procedure of deriving at least one piece of first compressed projection data by making resolution of at least one piece of first projection data of at least one energy band among the plurality of pieces of projection data relatively lower than resolution of projection data of a plurality of other energy bands other than the at least one energy band; and a procedure of transmitting the at least one piece of first compressed projection data and the projection data of the plurality of other energy bands to the second data processing apparatus.

In the data processing program according to the present disclosure, the program may further cause the computer to execute: in the second data processing apparatus, a procedure of acquiring the at least one piece of first compressed projection data and the projection data of the plurality of other energy bands transmitted by the data processing program according to the present disclosure; and a procedure of deriving first restored projection data by restoring the first compressed projection data based on the projection data of the plurality of other energy bands and the first compressed projection data.

According to the present disclosure, it is possible to efficiently transmit the projection data of three or more energy bands while ensuring reproducibility.

### BRIEF DESCRIPTION OF THE DRAWINGS

FIG. 1 is a diagram showing a schematic configuration of a data processing system according to an embodiment of the present disclosure.

FIG. 2 is a flowchart showing processing performed in the present embodiment.

FIG. 3 is a diagram for describing compression and restoration of first projection data.

FIG. 4 is a diagram showing a relationship between a first ratio and a second ratio.

### DETAILED DESCRIPTION

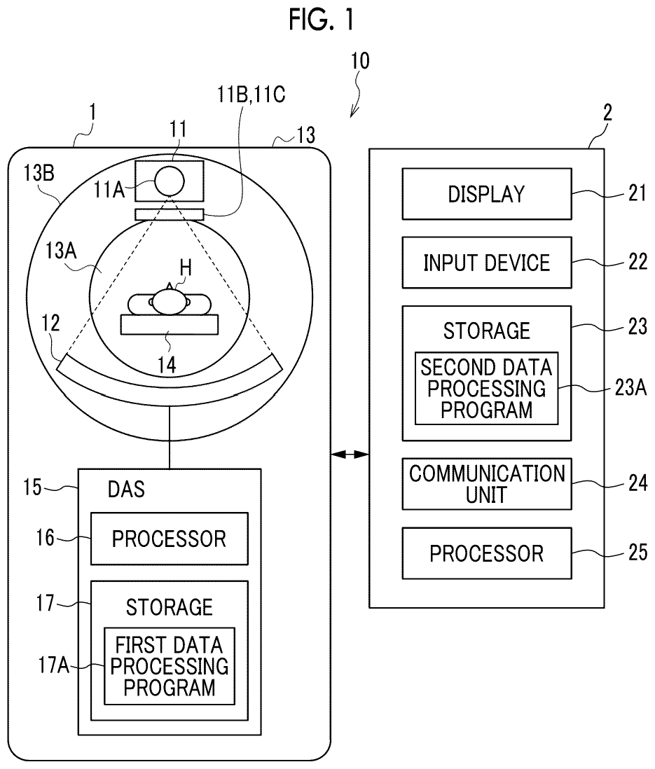

In the following, an embodiment of the present disclosure will be explained with reference to the drawings. FIG. 1 is a diagram showing a schematic configuration of a data processing system according to the present embodiment. As shown in FIG. 1, a data processing system 10 according to the present embodiment comprises a CT apparatus 1 and a console 2.

The CT apparatus 1 is a PCCT apparatus that detects radiation emitted from a radiation source and generates projection data for generating a tomographic image based on a detection signal corresponding to the number of photons of the radiation. In the present embodiment, a case where the radiation is X-rays will be described as an example. The CT apparatus 1 comprises an X-ray source 11, an X-ray detector 12, a gantry 13, a bed 14, and a data acquisition system (DAS) 15.

A circular opening portion 13A for disposing the bed 14 on which a subject H is placed is provided at the center of the gantry 13. In addition, the gantry 13 is provided with a rotating plate 13B, to which the X-ray source 11 and the X-ray detector 12 (hereinafter, simply referred to as a detector 12) are fixed in opposing positions, and a drive mechanism (not shown) for rotating the rotating plate 13B.

The X-ray source 11 includes an X-ray tube 11A, an X-ray filter 11B, and a bowtie filter 11C. The X-ray tube 11A generates X-rays and irradiates the subject H with the generated X-rays. The X-ray filter 11B adjusts the dose of the X-rays emitted from the X-ray tube 11A. The bowtie filter 11C optimizes an exposure dose by increasing the dose near the center and reducing the dose around the periphery in order to suppress the exposure dose in a peripheral portion.

The detector 12 has a detection surface in which a plurality of detection elements are arranged two-dimensionally, detects the X-rays transmitted through the subject H, and outputs a detection signal for each detection element. Therefore, it is possible to detect a detection signal of the radiation for each transmission position where the radiation transmits through the structure of the subject H. The detector 12 is an example of a photon counting detector of the present disclosure.

The photon counting detector is capable of measuring energy for each photon and outputting energy information for each of a plurality of energy bands as a detection signal. In the present embodiment, the detector 12 outputs detection signals for each of, for example, three energy bands. For example, the energy band can be 30 keV or more and less than 50 keV, 50 keV or more and less than 100 keV, and 100 keV or more, but the present invention is not limited thereto.

The DAS 15 collects the detection signal for each energy band output by the detector 12, generates projection data for each energy band at each position of the rotating plate 13B about a rotation axis based on the collected detection signals, and transmits the generated projection data to the console 2. The DAS 15 is an example of a first data processing apparatus of the present disclosure. For the projection data, a direction along the periphery of the rotating plate 13B is referred to as an X direction, and a direction along the rotation axis is referred to as a Y direction on the detection surface of the detector 12. In the present embodiment, first projection data P1, second projection data P2, and third projection data P3 are arranged in descending order of the energy band.

In the present embodiment, the DAS 15 has a processor 16 and a storage 17 in order to perform first data processing, which will be described below. As an example, the processor 16 is configured with a central processing unit (CPU) and a memory, such as a random access memory (RAM). The storage 17 is a data storage that stores a first data processing program 17A executed by the DAS 15. Examples of the storage 17 include a hard disk drive (HDD) and a solid state drive (SSD). The processor 16 loads the first data processing program 17A from the storage 17 into the memory, and executes the loaded program to perform the first data processing.

The console 2 comprises a display 21, an input device 22, a storage 23, a communication unit 24, and a processor 25. The console 2 is configured, for example, based on a personal computer, and a hardware configuration thereof is the same as a hardware configuration of a general computer. The display 21 is, for example, a liquid crystal display or the like and displays an operation screen, a captured tomographic image, and the like. The input device 22 is a device for an operator to input an operation instruction, and is configured with a keyboard, a mouse, and the like. The console 2 is an example of a second data processing apparatus of the present disclosure.

The storage 23 is a data storage that stores various programs such as a control program that controls each unit of the console 2. The various programs include an application program including a second data processing program 23A for causing the console 2 to function as the second data processing apparatus. Examples of the storage 23 include an HDD and an SSD. In addition, the storage 23 also temporarily stores the projection data acquired from the CT apparatus 1.

The communication unit 24 is a communication interface for performing communication between the CT apparatus 1 and the console 2. The communication unit 24 is connected to a network (not shown), such as a local area network (LAN) and/or a wide area network (WAN), and performs transmission control in accordance with a communication protocol defined in various types of wired or wireless communication standards.

As an example, the processor 25 is configured with a CPU and a memory such as a RAM. The processor 25 loads various programs including the second data processing program 23A from the storage 23 into the memory, and executes the loaded programs to perform the second data processing.

In addition, the processor 25 controls each unit of the CT apparatus 1 in accordance with an instruction of the operator input from the input device 22, and causes the CT apparatus 1 to perform the imaging of the subject H. In addition, the processor 25 performs reconstruction processing of generating a tomographic image by reconstructing the tomographic image based on the projection data that is acquired from the CT apparatus 1.

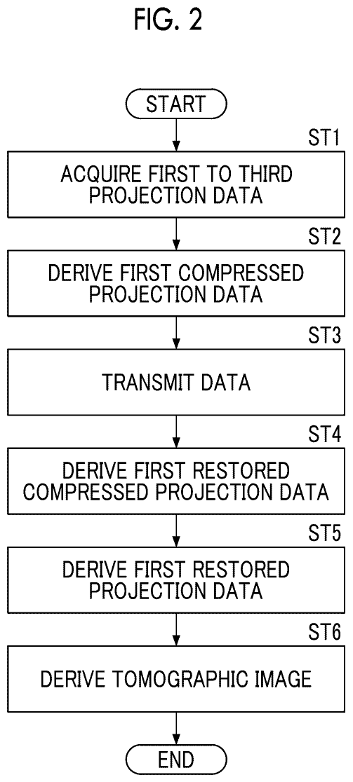

Next, processing performed in the present embodiment will be described. FIG. 2 is a flowchart showing the processing performed in the present embodiment. Here, the first data processing performed by the DAS 15 in the CT apparatus 1 and the second data processing performed by the processor 25 of the console 2 will be described. First, in the CT apparatus 1, the first to third projection data P1 to P3 of the three energy bands at each position around the rotation axis of the detector 12 are acquired based on the detection signal in each pixel by the detector 12 (Step ST1).

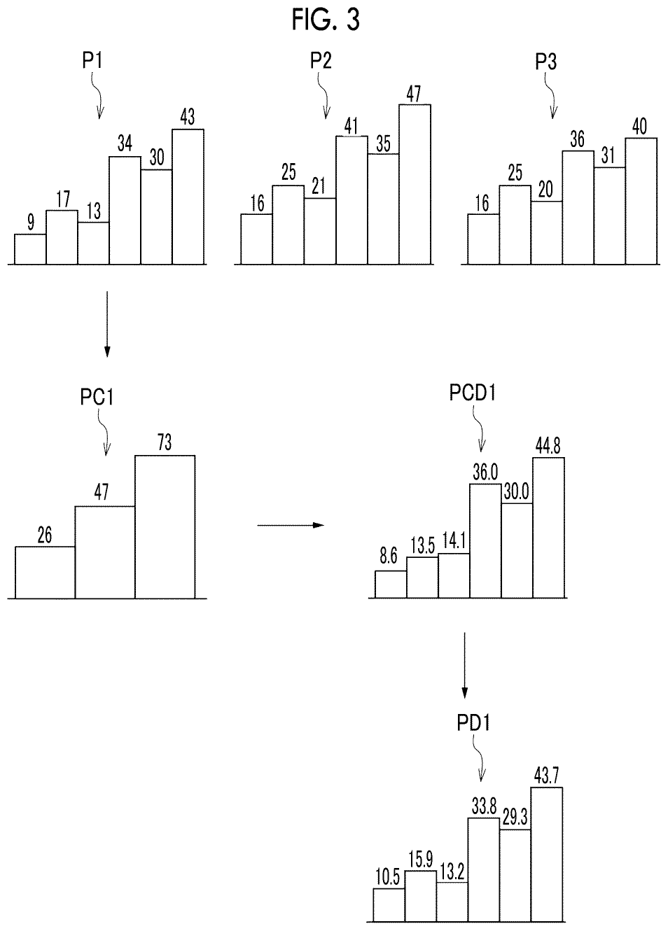

Then, the DAS 15 derives a first compressed projection data PC1 by making resolution of the first projection data P1 in the highest energy band relatively lower than resolution of the second projection data P2 and the third projection data P3 (Step ST2). FIG. 3 is a diagram for describing a derivation of the first compressed projection data. In FIG. 3, for the first projection data P1 to the third projection data P3, values of detection signals, that is, pixel values, of six pixels arranged in the X direction of each projection data are shown.

The DAS 15 derives the first compressed projection data PC1 obtained by compressing the first projection data P1 in the X direction by adding the pixel values of two adjacent pixels for the first projection data P1 to make the resolution of the first projection data P1 in the X direction 1/2. Specifically, as shown in FIG. 3, since the pixel values of two pixels on the left side of the first projection data P1 are 9 and 17, respectively, the leftmost pixel value of the first compressed projection data PC1 is 26. In addition, since the pixel values of two central pixels of the first projection data P1 are 13 and 34, respectively, the central pixel value of the first compressed projection data PC1 is 47. In addition, since the pixel values of two pixels on the right side of the first projection data P1 are 30 and 43, respectively, the pixel value of the rightmost pixel of the first compressed projection data PC1 is 73.

A direction in which the first projection data P1 is compressed may be the Y direction or both the X direction and the Y direction.

The DAS 15 transmits the first compressed projection data PC1, the second projection data P2, and the third projection data P3 to the console 2 (data transmission: Step ST3). The second projection data P2 and the third projection data P3 are not compressed. Steps ST1 to ST3 correspond to the first data processing.

The processor 25 of the console 2 derives first restored projection data PD1 by restoring the first compressed projection data PC1 based on the second projection data P2 and the third projection data P3. In the present embodiment, the processor 25 derives the first restored projection data PD1 by restoring the first compressed projection data PC1 based on a relationship between a first ratio R1 of the second projection data P2 to the third projection data P3 and a second ratio R2 of the first projection data P1 to the third projection data P3.

In the following, the relationship between the first ratio R1 and the second ratio R2 will be described. In the present embodiment, a large number of first projection data P1, second projection data P2, and third projection data P3 are acquired in advance. The processor 25 derives the first ratio R1 (=P2/P3) and the second ratio R2 (=P1/P3) for each pixel value of the first projection data P1, the second projection data P2, and the third projection data P3 which are acquired at the same time of imaging.

The magnitude of the energy of the energy bands of the first projection data P1, the second projection data P2, and the third projection data P3 is P1>P2>P3. Therefore, the first ratio R1 and the second ratio R2 represent the quality of X-rays transmitted through the subject H and emitted to the detector 12. Here, the X-rays emitted from the X-ray source 11 are attenuated differently depending on the thickness and composition of the subject H, and thus X-rays with different qualities (that is, spectral hardness) are detected in the detector 12.

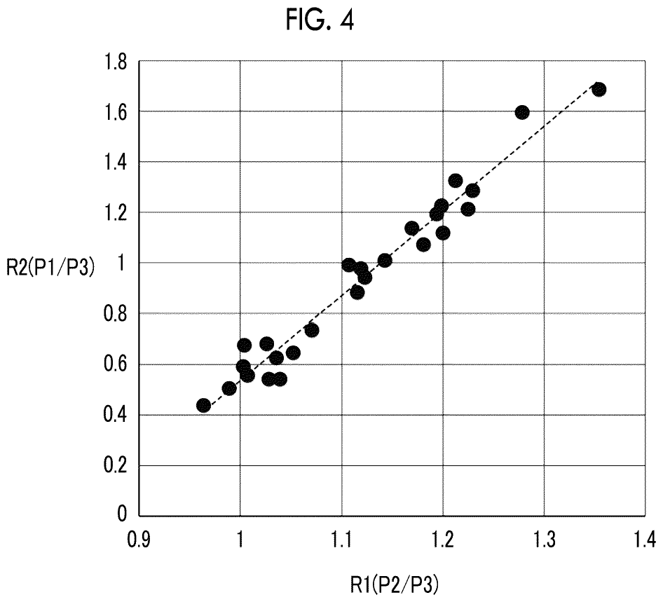

FIG. 4 is a diagram showing an example of a relationship between the first ratio R1 and the second ratio R2. In FIG. 4, a horizontal axis represents the first ratio R1, and a vertical axis represents the second ratio R2. As shown in FIG. 4, the relationship between the first ratio R1 and the second ratio R2 is generally represented by a linear function. Therefore, the relationship between the first ratio R1 and the second ratio R2 is represented by the following Expression (1). In Expression (1), a is the slope of the straight line in FIG. 4, and b is the value of the intersection point between the straight line and the R2 axis.

R2=a×R1+b (1)

From the relationship shown in FIG. 4, it can be seen that the second ratio R2 also becomes large in a case where the first ratio R1 is large (that is, the radiation quality is hard). The relationship between the first ratio R1 and the second ratio R2 is derived in advance and stored in the storage 23.

The relationship between the first ratio R1 and the second ratio R2 is derived by actually measuring the first projection data P1, the second projection data P2, and the third projection data P3. Thus, the relationship between the first ratio R1 and the second ratio R2 may be represented not only by a linear function but also by a multidimensional function, a non-linear function, or the like.

The processor 25 restores the first compressed projection data with reference to the relationship between the first ratio R1 and the second ratio R2 and derives first restored compressed projection data PCD1 (Step ST4). In a case where the first ratio R1 and the second ratio R2 have the relationship shown in the above Expression (1), Expression (1) can be transformed into the following Expression (2).

(P1/P3)=a×(P2/P3)+b (2)

Therefore, a pixel value of the first restored compressed projection data PCD1 can be derived by the following Expression (3).

PCD1=a×P2+b×P3 (3)

For example, in a case where a=3.32 and b=−2.78, as shown in FIG. 3, the pixel values of the first restored compressed projection data PCD1 are derived as 8.6, 13.5, 14.1, 36.0, 30.0, and 44.8 from left to right.

Next, the processor 25 derives the first restored projection data PD1 by correcting the first restored compressed projection data PCD1 to match the first compressed projection data PC1 (Step ST5). Here, as shown in FIG. 3, two pixel values on the left side of the first restored compressed projection data PCD1 are 8.6 and 13.5, and the total value of these is 22.1. Comparing the total value of 22.1 with the leftmost pixel value of 26 in the first compressed projection data PC1, the pixel value of the first compressed projection data PC1 is 18% larger. Therefore, the processor 25 corrects the pixel values on the left side of the first restored projection data PD1 to 10.1 and 15.9, respectively, by increasing the two pixel values of the first restored compressed projection data PCD1 on the left side by 18%.

Similarly, the processor 25 corrects the two central pixel values 14.1 and 36.0 of the first restored compressed projection data PCD1 to 13.2 and 33.8 based on the central pixel value 47 of the first compressed projection data PC1. Further, the processor 25 corrects the two pixel values 30.0 and 44.8 on the right side of the first restored compressed projection data PCD1 to 29.3 and 43.7, based on the pixel value 73 on the right side of the first compressed projection data PC1. Steps ST4 and ST5 correspond to the second data processing.

Then, the processor 25 derives a tomographic image by performing reconstruction using the first restored projection data PD1, the second projection data P2, and the third projection data P3 (Step ST6), and ends the processing. That is, for the energy band of the first projection data P1, the processor 25 generates a tomographic image by using the first restored projection data PD1. For the energy band of the second projection data P2, a tomographic image is generated by using the second projection data P2. For the energy band of the third projection data P3, a tomographic image is generated by using the third projection data P3.

As described above, in the present embodiment, at least one piece of first compressed projection data PC1 is derived by making the resolution of at least one piece of first projection data P1 of at least one energy band of the plurality of pieces of projection data P1 to P3 relatively lower than the resolution of the projection data of the other energy bands, and the plurality of pieces of projection data (that is, the second projection data P2 and the third projection data P3) other than at least one piece of first compressed projection data PC1 among the first compressed projection data PC1 and the plurality of pieces of projection data are transmitted to the console 2. For this reason, it is possible to reduce the amount of data of the projection data transmitted from the CT apparatus 1 to the console 2. Therefore, it is possible to efficiently transmit the projection data of three or more energy bands.

In addition, in the present embodiment, the first compressed projection data PC1 is restored based on at least one piece of the first compressed projection data PC1 and the plurality of pieces of projection data (that is, the second projection data P2 and the third projection data P3) to derive the first restored projection data PD1. Therefore, it is possible to efficiently transmit the projection data of three or more energy bands while ensuring reproducibility.

In the embodiment described above, the relationship between the first ratio R1 and the second ratio R2 is derived by the expression, but the present invention is not limited thereto. For example, a relationship between the first projection data P1 and the second projection data P2 and the third projection data P3 may be derived in advance as a look-up table and stored in the storage 23, and the first projection data P1 may be derived by referring to the look-up table.

In addition, in the embodiment described above, the first projection data P1 of the highest energy band is compressed, but the present disclosure is not limited thereto. The third projection data P3 of the lowest energy band may be compressed, or the second projection data P2 of an intermediate energy band may be compressed.

In addition, in the embodiment described above, the photon counting detector outputs detection signals of three energy bands, but the present invention is not limited thereto. The detection signals of three or more energy bands may be derived. In this case as well, in the DAS 15, the projection data of at least one energy band among the plurality of energy bands may be compressed and transmitted to the console 2. In the console 2, a relationship between the compressed projection data of the energy band and the projection data of the other energy bands may be derived in advance, and the compressed projection data of the energy band may be restored by referring to the relationship.

In addition, in the embodiment described above, the processors 16 and 25 include, in addition to a CPU that is a general-purpose processor that executes software (program) to function as various processing units, a programmable logic device (PLD) of which a circuit configuration can be changed after manufacturing, such as a field-programmable gate array (FPGA), and a dedicated electric circuit that is a processor having a circuit configuration dedicatedly designed for executing specific processing, such as an ASIC.

The various pieces of processing may be executed by one of the various processors or a combination of two or more processors of the same type or different types (for example, a plurality of FPGAs and a combination of CPU and FPGA). Further, a plurality of processing units may be composed of one processor. As an example where a plurality of processing units are composed of one processor, there is a form in which a processor that realizes all functions of a system including a plurality of processing units into one integrated circuit (IC) chip is used, such as a system on a chip (SOC).

Hereinafter, the supplementary notes of the present disclosure will be described.

### Supplementary Note 1

A data processing system comprising:
a first data processing apparatus that processes projection data from a photon counting detector which detects radiation emitted from a radiation source and transmitted through a subject and which outputs a plurality of pieces of projection data assigned to each of three or more energy bands corresponding to the number of photons of the radiation; and
a second data processing apparatus,
wherein the first data processing apparatus includes at least one first processor,
the first processor derives at least one piece of first compressed projection data by making resolution of at least one piece of first projection data of at least one energy band among the plurality of pieces of projection data relatively lower than resolution of projection data of a plurality of other energy bands other than the at least one energy band, and
   transmits the at least one piece of first compressed projection data and the projection data of the plurality of other energy bands to the second data processing apparatus,
the second data processing apparatus includes at least one second processor, and
the second processor acquires the at least one piece of first compressed projection data and the projection data of the plurality of other energy bands, and
   derives first restored projection data by restoring the first compressed projection data based on the projection data of the plurality of other energy bands and the first compressed projection data.

### Supplementary Note 2

The data processing system according to supplementary note 1,
wherein an energy band of the first projection data is relatively higher in energy than an energy band of at least one piece of projection data among the projection data of the plurality of other energy bands.

### Supplementary Note 3

The data processing system according to supplementary note 1 or 2,
wherein the second processor derives first restored compressed projection data by restoring the first compressed projection data based on a relationship between the first projection data and the projection data of the plurality of other energy bands, which is derived in advance, and
   derives the first restored projection data by correcting the first restored compressed projection data to match the first compressed projection data.

### Supplementary Note 4

The data processing system according to supplementary note 3,
wherein the photon counting detector outputs three pieces of projection data assigned to each of three energy bands,
the first processor derives the first compressed projection data from the first projection data among the three pieces of projection data, and transmits second projection data and third projection data as the projection data of the plurality of other energy bands other than the first projection data among the three pieces of projection data together with the first compressed projection data to the second data processing apparatus, and
the second processor derives the first restored compressed projection data by restoring the first compressed projection data based on a relationship between the first projection data, the second projection data, and the third projection data.

### Supplementary Note 5

The data processing system according to supplementary note 4,
wherein the second processor derives the first restored compressed projection data by restoring the first compressed projection data based on a relationship between a first ratio of the second projection data to the third projection data and a second ratio of the first projection data to the third projection data.

### Supplementary Note 6

A data processing method in a data processing system including a first data processing apparatus that processes projection data from a photon counting detector which detects radiation emitted from a radiation source and transmitted through a subject and which outputs a plurality of pieces of projection data assigned to each of three or more energy bands corresponding to the number of photons of the radiation and a second data processing apparatus, the method comprising:
causing a computer to, in the first data processing apparatus, derive at least one piece of first compressed projection data by making resolution of at least one piece of first projection data of at least one energy band among the plurality of pieces of projection data relatively lower than resolution of projection data of a plurality of other energy bands other than the at least one energy band, and
   transmit the at least one piece of first compressed projection data and the projection data of the plurality of other energy bands to the second data processing apparatus.

### Supplementary Note 7

A data processing method comprising:
causing the computer to, in the second data processing apparatus, acquire the at least one piece of first compressed projection data and the projection data of the plurality of other energy bands that are transmitted by the data processing method according to supplementary note 6, and
   derive first restored projection data by restoring the first compressed projection data based on the projection data of the plurality of other energy bands and the first compressed projection data.

### Supplementary Note 8

A data processing program that causes a computer to execute data processing in a data processing system including a first data processing apparatus that processes projection data from a photon counting detector which detects radiation emitted from a radiation source and transmitted through a subject and which outputs a plurality of pieces of projection data assigned to each of three or more energy bands corresponding to the number of photons of the radiation and a second data processing apparatus, the data processing program causing the computer to execute:
in the first data processing apparatus, a procedure of deriving at least one piece of first compressed projection data by making resolution of at least one piece of first projection data of at least one energy band among the plurality of pieces of projection data relatively lower than resolution of projection data of a plurality of other energy bands other than the at least one energy band; and
   a procedure of transmitting the at least one piece of first compressed projection data and the projection data of the plurality of other energy bands to the second data processing apparatus.

### Supplementary Note 9

A data processing program that causes a computer to execute:
in the second data processing apparatus, a procedure of acquiring the at least one piece of first compressed projection data and the projection data of the plurality of other energy bands that are transmitted by the data processing program according to supplementary note 8; and
a procedure of deriving first restored projection data by restoring the first compressed projection data based on the projection data of the plurality of other energy bands and the first compressed projection data.

What is claimed is:

1. A data processing system comprising:
a first data processing apparatus that processes projection data from a photon counting detector which detects radiation emitted from a radiation source and transmitted through a subject and which outputs a plurality of pieces of projection data assigned to each of three or more energy bands corresponding to the number of photons of the radiation; and
a second data processing apparatus,
wherein the first data processing apparatus includes at least one first processor,
the first processor derives at least one piece of first compressed projection data by making resolution of at least one piece of first projection data of at least one energy band among the plurality of pieces of projection data relatively lower than resolution of projection data of a plurality of other energy bands other than the at least one energy band, and
transmits the at least one piece of first compressed projection data and the projection data of the plurality of other energy bands to the second data processing apparatus,
the second data processing apparatus includes at least one second processor, and
the second processor acquires the at least one piece of first compressed projection data and the projection data of the plurality of other energy bands, and
derives first restored projection data by restoring the first compressed projection data based on the projection data of the plurality of other energy bands and the first compressed projection data.

2. The data processing system according to claim 1,
wherein an energy band of the first projection data is relatively higher in energy than an energy band of at least one piece of projection data among the projection data of the plurality of other energy bands.

3. The data processing system according to claim 1,
wherein the second processor derives first restored compressed projection data by restoring the first compressed projection data based on a relationship between the first projection data and the projection data of the plurality of other energy bands, which is derived in advance, and
derives the first restored projection data by correcting the first restored compressed projection data to match the first compressed projection data.

4. The data processing system according to claim 2,
wherein the second processor derives first restored compressed projection data by restoring the first compressed projection data based on a relationship between the first projection data and the projection data of the plurality of other energy bands, which is derived in advance, and
derives the first restored projection data by correcting the first restored compressed projection data to match the first compressed projection data.

5. The data processing system according to claim 3,
wherein the photon counting detector outputs three pieces of projection data assigned to each of three energy bands,
the first processor derives the first compressed projection data from the first projection data among the three pieces of projection data, and transmits second projection data and third projection data as the projection data of the plurality of other energy bands other than the first projection data among the three pieces of projection data together with the first compressed projection data to the second data processing apparatus, and
the second processor derives the first restored compressed projection data by restoring the first compressed projection data based on a relationship between the first projection data, the second projection data, and the third projection data.

6. The data processing system according to claim 4,
wherein the photon counting detector outputs three pieces of projection data assigned to each of three energy bands,
the first processor derives the first compressed projection data from the first projection data among the three pieces of projection data, and transmits second projection data and third projection data as the projection data of the plurality of other energy bands other than the first projection data among the three pieces of projection data together with the first compressed projection data to the second data processing apparatus, and
the second processor derives the first restored compressed projection data by restoring the first compressed projection data based on a relationship between the first projection data, the second projection data, and the third projection data.

7. The data processing system according to claim 5,
wherein the second processor derives the first restored compressed projection data by restoring the first compressed projection data based on a relationship between a first ratio of the second projection data to the third projection data and a second ratio of the first projection data to the third projection data.

8. The data processing system according to claim 6,
wherein the second processor derives the first restored compressed projection data by restoring the first compressed projection data based on a relationship between a first ratio of the second projection data to the third projection data and a second ratio of the first projection data to the third projection data.

9. A data processing method in a data processing system including a first data processing apparatus that processes projection data from a photon counting detector which detects radiation emitted from a radiation source and transmitted through a subject and which outputs a plurality of pieces of projection data assigned to each of three or more energy bands corresponding to the number of photons of the radiation and a second data processing apparatus, the method comprising:
causing a computer to, in the first data processing apparatus, derive at least one piece of first compressed projection data by making resolution of at least one piece of first projection data of at least one energy band among the plurality of pieces of projection data relatively lower than resolution of projection data of a plurality of other energy bands other than the at least one energy band, and
transmit the at least one piece of first compressed projection data and the projection data of the plurality of other energy bands to the second data processing apparatus.

10. A data processing method comprising:
causing the computer to, in the second data processing apparatus, acquire the at least one piece of first compressed projection data and the projection data of the plurality of other energy bands that are transmitted by the data processing method according to claim 9, and
derive first restored projection data by restoring the first compressed projection data based on the projection data of the plurality of other energy bands and the first compressed projection data.

11. A non-transitory computer-readable storage medium that stores a data processing program that causes a computer to execute data processing in a data processing system including a first data processing apparatus that processes projection data from a photon counting detector which detects radiation emitted from a radiation source and transmitted through a subject and which outputs a plurality of pieces of projection data assigned to each of three or more energy bands corresponding to the number of photons of the radiation and a second data processing apparatus, the data processing program causing the computer to execute:
in the first data processing apparatus, a procedure of deriving at least one piece of first compressed projection data by making resolution of at least one piece of first projection data of at least one energy band among the plurality of pieces of projection data relatively lower than resolution of projection data of a plurality of other energy bands other than the at least one energy band; and
a procedure of transmitting the at least one piece of first compressed projection data and the projection data of the plurality of other energy bands to the second data processing apparatus.

12. A non-transitory computer-readable storage medium that stores a data processing program that causes a computer to execute:
in the second data processing apparatus, a procedure of acquiring the at least one piece of first compressed projection data and the projection data of the plurality of other energy bands that are transmitted by the data processing program according to claim 8; and
a procedure of deriving first restored projection data by restoring the first compressed projection data based on the projection data of the plurality of other energy bands and the first compressed projection data.
